(19) United States
(12) Patent Application Publication
(10) Pub. No.: US 2014/0011814 A1
(43) Pub. Date: Jan. 9, 2014

(54) DIAMINE AND MEGLUMINE SALT FORMS OF FATTY ACIDS

(71) Applicants: Banavara L. MYLARI, Lutz, FL (US); Frank C. SCIAVOLINO, Waterford, CT (US)

(72) Inventors: Banavara L. MYLARI, Lutz, FL (US); Frank C. SCIAVOLINO, Waterford, CT (US)

(73) Assignee: THETIS PHARMACEUTICALS LLC, Southport, CT (US)

(21) Appl. No.: 13/841,667

(22) Filed: Mar. 15, 2013

Related U.S. Application Data
(60) Provisional application No. 61/668,517, filed on Jul. 6, 2012.
(60) Provisional application No. 61/670,384, filed on Jul. 11, 2012.

(51) Int. Cl. C07C 57/03; C07D 295/027

(52) U.S. Cl. CPC C07C 57/03; C07D 295/027
USPC 514/252.12; 544/358

(57) ABSTRACT

Provided herein are diamine salts of eicosapentaenoic acid and docosahexaenoic acid, processes for the preparation of such compounds, pharmaceutical compositions comprising such compounds, and the uses of such compounds as agents for treating dyslipidemia, cardiovascular diseases such as cardiac arrhythmia, cardiac ischemia, myocardial infarction, cardiomyopathy, and stroke and obesity.

### RELATED APPLICATIONS

This application claims priority to U.S. Provisional Application No. 61/668,517, filed Jul. 6, 2012, and U.S. Provisional Application No. 61/670,384, filed Jul. 11, 2012. The contents of both of these applications are incorporated herein by reference in their entireties.

### BACKGROUND OF THE INVENTION

Of the many health issues plaguing humans, obesity and overall poor cardiovascular to health are among the most distressing, due to the fact that these diseases can lead to a number of complications. For example, obesity is often associated with hyperlipidemia, which is an elevation of lipids in the blood. These lipids include triglycerides, cholesterol, cholesterol esters, and phospholipids. Specifically, elevated levels of triglycerides in the blood is known as hypertriglyceridemia. Although a certain amount of triglycerides are necessary for good health, increased triglyceride levels are often associated with increased risk of heart disease. Overall, hyperlipidemia is associated with a number of disease states, including coronary artery disease, angina pectoris, carotid artery disease, strokes, cerebral arteriosclerosis, and xanthoma.

Accordingly, there remains a need for treating obesity, cardiovascular disease, and related indications.

### SUMMARY OF THE INVENTION

Provided herein are compounds useful in the treatment of obesity, cardiovascular disease, and related indications such as such as cardiac arrhythmia, cardiac ischemia, myocardial infarction, cardiomyopathy, and stroke.

In an embodiment, provided herein are compounds of the Formula A, B, C, D, E or F:

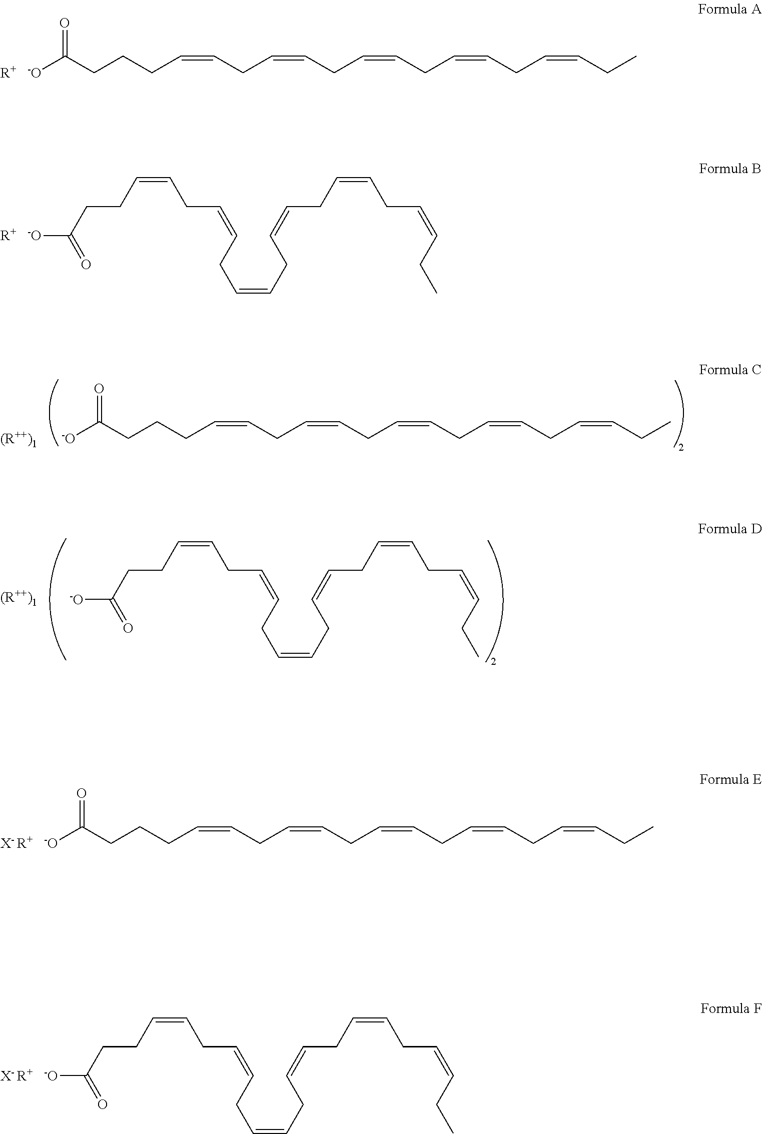

wherein, for Formulas A and B, R⁺ represents a piperidine or diamine group, wherein the nitrogen of the piperidine and one of the nitrogens of the diamine are protonated;

for Formulas C and D, R⁺⁺ represents a diamine group, wherein two of the nitrogens of the diamine are protonated; and

for Formulas E and F, R⁺ represents a diamine group, wherein two of the nitrogens of the diamine are protonated, and X⁻ is an anion of a pharmaceutically acceptable acid compound.

In another embodiment, the compounds provided herein are of the Formula I, II, III, IV, V, VI, VII and VIII:

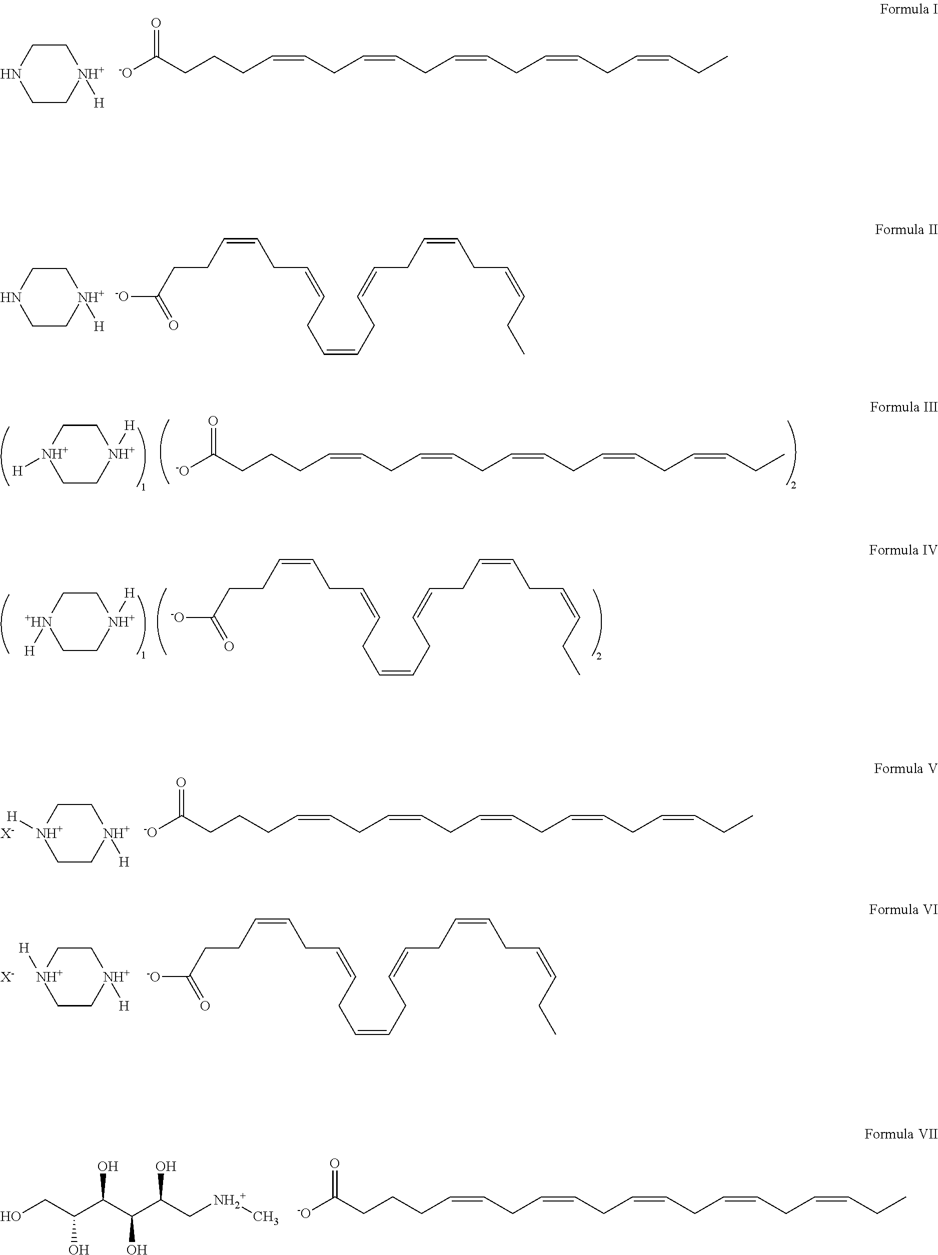

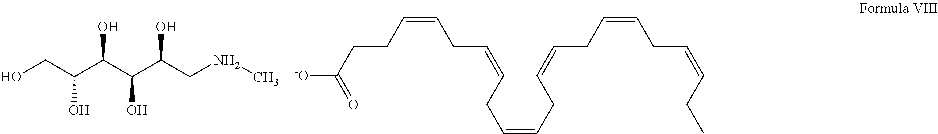

wherein X⁻ is as defined below.

The compounds of Formula A, B, C, D, E, F, I, II, III, IV, V, VI, VII and VIII are referred to herein as the “compounds of the invention.”

In an embodiment, provided herein is a pharmaceutical composition comprising a compound of the invention, and a pharmaceutically acceptable carrier. In another embodiment, to provided herein is a kit comprising a) a unit dosage comprising a compound of the invention, and b) instructions on how to use the kit; and at least one container for holding the unit dosage forms.

The compounds of the invention can be used to treat obesity, cardiovascular disease, and related indications, in a subject in need thereof. In one aspect, provided herein is a method of treating hyperlipidemia, comprising administering to a subject in need thereof an effective amount of a compound of the invention. In another aspect, provided herein is a method of treating hypertriglyceridemia, comprising administering to a subject in need thereof an effective amount of a compound the invention. In another aspect, provided herein is a method of treating dyslipidemia, comprising administering to a subject in need thereof an effective amount of a compound of the invention. In still another aspect, provided herein is a method of treating cardiovascular disease, comprising administering to a subject in need thereof an effective amount of a compound of the invention. The cardiovascular disease can be cardiac arrhythmia, cardiac ischemia, myocardial infarction, cardiomyopathy, or stroke. In an embodiment, the arrhythmia is an atrial fibrillation. In another aspect, provided herein is a method of treating obesity, comprising administering to a subject in need thereof an effective amount of a compound of the invention. In one embodiment of the above methods, the subject is human.

In another aspect, provided herein is a method of treating prediabetes, comprising administering to a subject in need thereof an effective amount of a compound of the invention.

In still another aspect, provided herein is a method of treating atherosclerosis, comprising administering to a subject in need thereof an effective amount of a compound of the invention.

Also provided herein is a method of lowering triglycerides in a subject in need thereof, comprising administering to the subject an effective amount of a compound of the invention.

In another embodiment, provided herein is a method of treating atrial fibrillation, or reducing the probability of an occurrence of an atrial fibrillation, comprising administering to a subject in need thereof an effective amount of a compound of the invention.

### DETAILED DESCRIPTION OF THE INVENTION

Epidemiological and clinical evidence suggests that an increased intake of ω-3 polyunsaturated fatty acids (PUFAs) protects against mortality from coronary artery diseases. PUFAs include eicosapentaenoic acid (EPA) and docosahexaenoic acid (DHA). It is widely established that PUFAs protect against and can terminate ischemic ventricular arrhythmias (Billman et al. Circulation. 1999, 99, 2452-2457 and Kang et al. Am. J. Clin. Nutr. 2002, 71, 202S-207S). In particular, it is known that EPA is a promising treatment for prevention of major coronary events. PUFAs have multiple biological functions through lipid-dependent and lipid-independent mechanisms. EPA and mixtures of EPA and DHA have been shown to ameliorate triglycerides (TGs) lipid levels in patients with very high TGs. Also, EPA is shown to increase adiponectin secretion both in obese animals and obese human subjects (Itoh et al. Arteroscler. Thromb. Vasc. Biol. 2007, 27, 1918-1925). Increased adiponectin levels are beneficial in regulating both lipid and glucose metabolism in animals as well as in humans.

In one aspect, provided herein are compounds of the Formula A, B, C, D, E or F:

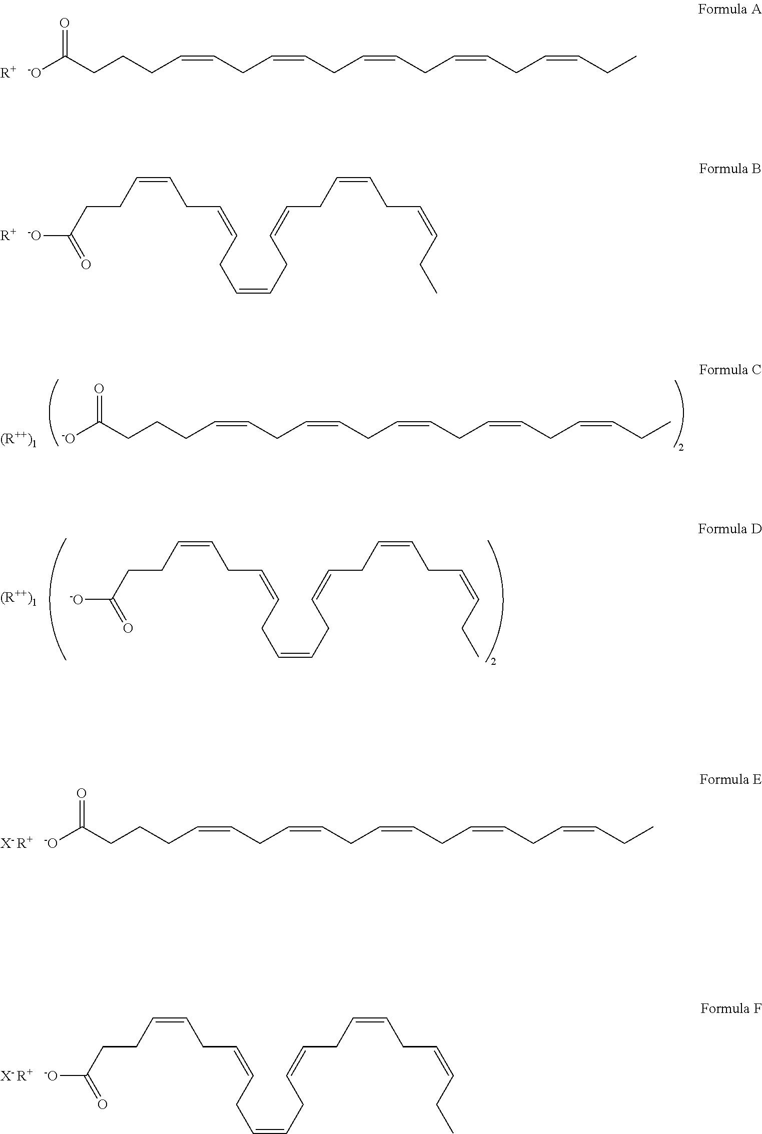

wherein, for Formulas A and B, R⁺ represents a piperidine or diamine group, wherein the nitrogen of the piperidine and one of the nitrogens of the diamine are protonated;

for Formulas C and D, R⁺⁺ represents a diamine group, wherein two of the nitrogens of the diamine are protonated; and

for Formulas E and F, R⁺ represents a diamine group, wherein two of the nitrogens of the diamine are protonated, and X⁻ is an anion of a pharmaceutically acceptable acid compound.

As used herein, the term “diamine” refers to an organic compound composed of two amino groups. The diamine can be any diamine composed of primary amines, secondary amines, or a combination of a primary amine(s) and a secondary amine(s). The diamine can be a cyclic diamine, such as piperazine, diaminocyclohexane, diphenylethylenediamine, 1,8-diaminonaphthalene, 4,4′-diaminobiphenyl, N,N′-di-2-butyl-1,4-phenylenediamine, dimethyl-4-phenylenediamine, p-xylylenediamine (PXD), m-xylylenediamine (MXD), o-xylylenediamine (OXD), p-phenylenediamine (PPD), 2,5-diaminotoluene, m-phenylenediamine (MPD), o-phenylenediamine (OPD). The diamine can also be an acyclic amine, such as ethylenediamine (1,2-diaminoethane), ethambutol, tetramethylethylenediamine (TMEDA or TEMED), 1,3-diaminopropane (propane-1,3-diamine), putrescine (butane-1,4-diamine), cadaverine (pentane-1,5-diamine), hexamethylenediamine (hexane-1,6-diamine), or 1,2-diaminopropane.

In another aspect, provided herein are piperazine salts of EPA and DHA, as well as meglumine salts of EPA and DHA.

In one aspect, provided herein is a compound of Formula I:

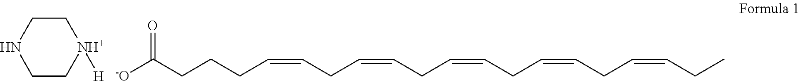

In another aspect, provided herein is a compound of Formula II:

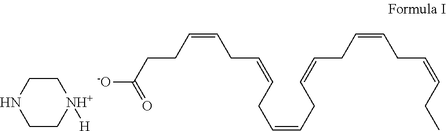

The compound of Formula I and II are, respectively, the mono-salt of EPA with piperazine and the mono-salt of DHA with piperazine.

In another aspect, provided herein are compounds of the Formula III and Formula IV:

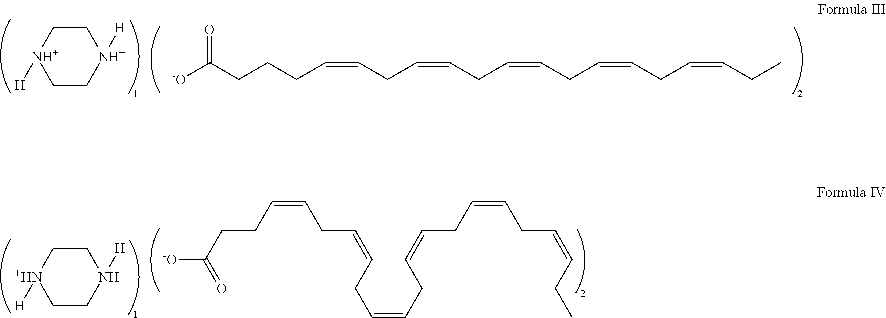

The compounds of Formula III and IV are, respectively, the di-salt of EPA with piperazine and the di-salt of DHA with piperazine.

In still another aspect, provided herein are compounds of the Formula V and VI:

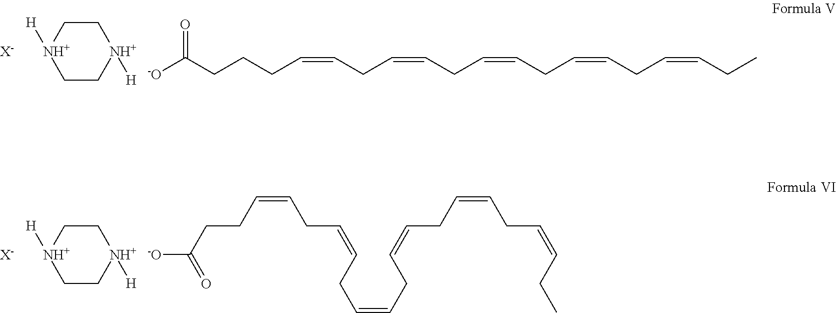

wherein X⁻ is a pharmaceutically acceptable counter anion.

The pharmaceutically acceptable counter anion can derived from acid compounds listed in Table 1, pp 406-407, Handbook of Pharmaceutical Salts, P. Heinrich Stahl Camille G. Wermuth (Eds.). In an embodiment, the pharmaceutically acceptable counter anion is selected from mineral acids, such as hydrochloric acid, hydrobromic acid, and phosphoric acid. In another embodiment, the pharmaceutically acceptable counter anion is selected from carboxylic acids, poly-carboxylic acids, and poly-hydroxy carboxylic acids, such as acetic acid, propionic acid, succinic acid, maleic acid, malic acid, tartaric acid, lactic acid, citric acid, and benzoic acid. In another embodiment, the pharmaceutically acceptable counter anion is selected from sulfonic acids and hydroxyl-sulfonic acids, including, but not limited to, methanesulfonic acid, isethionic acid, ethanesulfonic acid, 2-hydroxy-ethanesulfonic acid, and benzenesulfonic acid. In another embodiment, the pharmaceutically acceptable counter anion is selected from amino acids, including, but not limited to, glycine, alanine, lysine, arginine, aspartic acid, or glutamic acid. In another embodiment, X⁻ is an omega-3 polyunsaturated acid, such as eicosapentaenoic acid or docosahexaenoic acid.

In an embodiment, X⁻ is mandelic acid.

In an embodiment, provided herein are the following compounds: the hydrochloride salt of Formula V, the hydrobromide salt of Formula V, the phosphate salt of Formula V, and the sulfate salt of Formula V.

In another embodiment, provided herein are the following compounds: the hydrochloride salt of Formula VI, the hydrobromide salt of Formula VI, the phosphate salt of Formula VI, and the sulfate salt of Formula VI.

The present invention also relates to compounds of the Formula V and Formula VI wherein X⁻ is a pharmaceutically acceptable counter anion derived from naturally occurring amino acids. Examples of the amino acids include, but are not limited to, glycine, alanine, lysine, and glutamic acid.

In another aspect, provided herein are Compounds of the Formula VII and Formula VIII. These compounds, respectively, are EPA and DHA salts with meglumine:

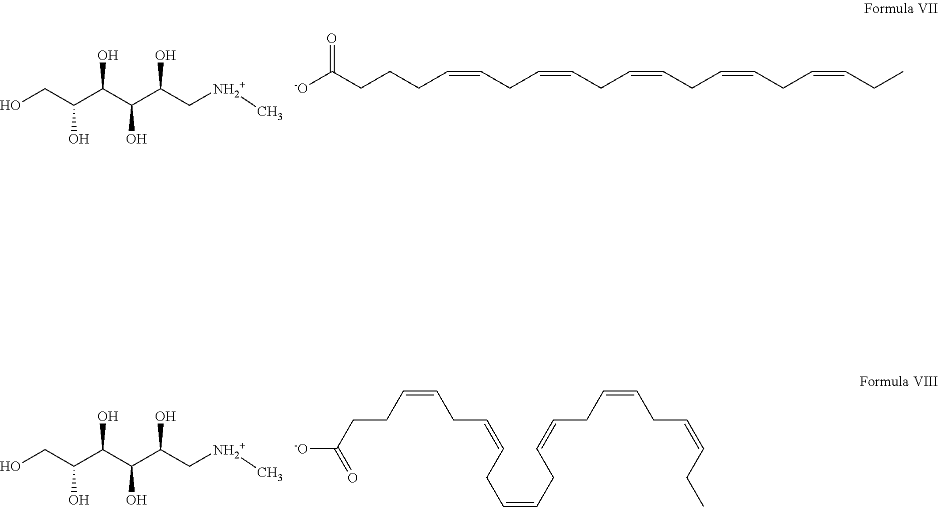

In another aspect, provided herein are compounds of Formulas IX-XIV

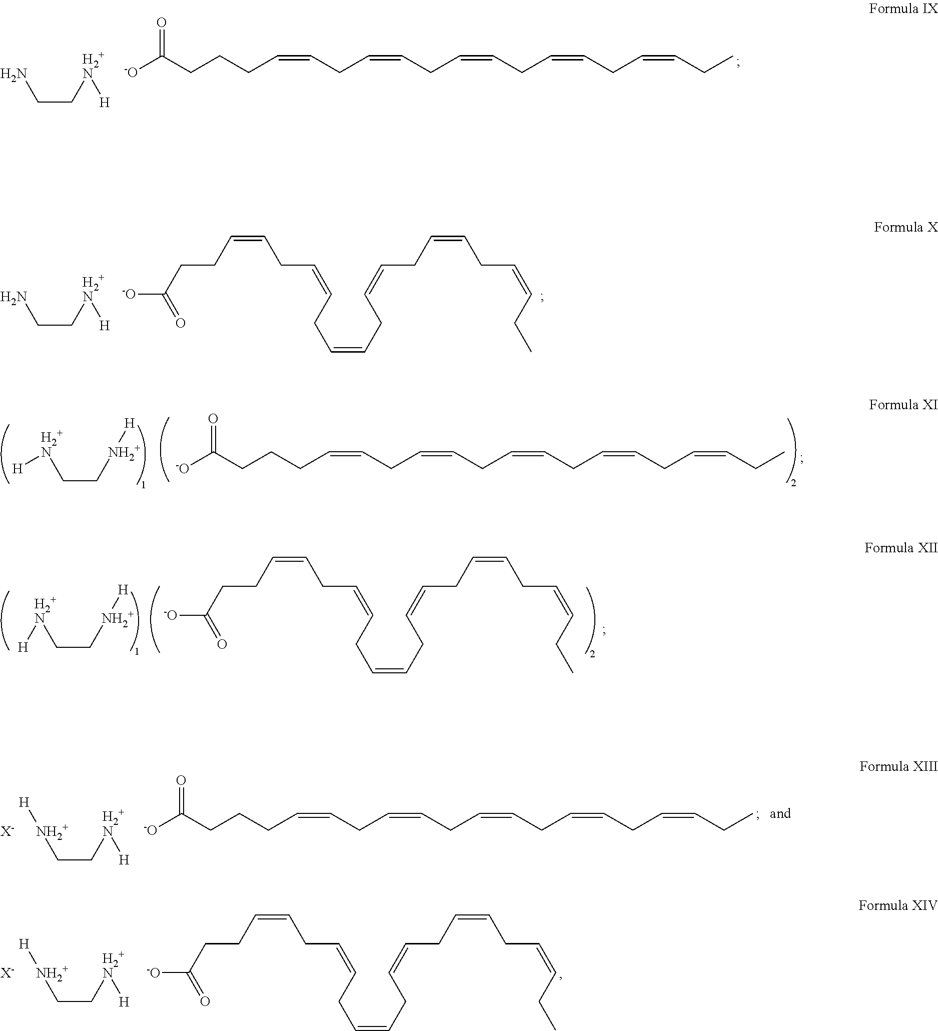

wherein X⁻ is a pharmaceutically acceptable counter anion as described above.

The compounds of the invention, e.g., compounds of Formulas I-XIV also include isomers and enantiomers wherever it is applicable.

It is well known in the art that highly water soluble medicinal preparations, when administered orally, result in efficient absorption of such preparations from the gastrointestinal is tract into systemic circulation. Another hallmark of such preparations is the rate at which they are absorbed into systemic circulation resulting in high concentration of the active agent or agents in the blood. Moreover, for delivery of xenobiotics via the intravenous route, they must be presented as a clear solution. PUFAs and esters of PUFAs are practically insoluble in water. In fact, they form soap-like emulsions when mixed with water. Therefore, the potential to derive optimum therapeutic benefits of PUFAs should be markedly facilitated by delivery of water soluble PUFAs. The compounds of the present invention are more water soluble to achieve high oral absorption and to enable the preparation of intravenous dosage forms.

### Methods of Treatment

In one aspect, provided herein is a method of treating obesity, cardiovascular disease, as well as related disorders, comprising administering to a subject in need thereof a compound of to the invention, e.g, compounds of Formulas I-XIV. In an embodiment, an effective amount of the compound is administered for treatment.

Hyperlipidemia is a condition generally characterized by an abnormal increase in serum lipids in the bloodstream and is an important risk factor in developing atherosclerosis and heart disease. Hyperlipidemia is usually classified as primary or secondary hyperlipidemia. Primary hyperlipidemia is generally caused by genetic defects, while secondary hyperlipidemia is generally caused by other factors, such as various disease states, drugs, and dietary factors. Alternatively, hyperlipidemia can result from both a combination of primary and secondary causes of hyperlipidemia. Elevated cholesterol levels are associated with a number of disease states, including coronary artery disease, angina pectoris, carotid artery disease, strokes, cerebral arteriosclerosis, and xanthoma.

An example of hyperlipidemia is hypertriglyceridemia, which is defined as an elevated level of triglycerides. Elevated triglycerides have been associated with atherosclerosis, even in the absence of high cholesterol levels. High triglycerides can also lead to pancreatitis in excessive concentrations.

As used herein, “obesity” refers to having a body weight more than about 30% greater than ideal body weight, as determined by a medical professional, and/or having a body mass index greater than about 27 as determined by a medical professional.

Accordingly, in one aspect, provided herein is a method of treating hyperlipidemia (e.g., hypertriglyceridemia) in a subject in need thereof, comprising administering to the subject an effective amount of a compound of the invention, e.g., compounds of Formulas I-XIV.

In another aspect, provided herein is a method of treating cardiovascular diseases such as cardiac arrhythmia, cardiac ischemia, myocardial infarction, cardiomyopathy, and stroke in a subject in need thereof, comprising administering to the subject an effective amount of a compound of the invention, e.g., compounds of Formulas I-XIV. In an embodiment, the arrhythmia is an atrial fibrillation.

In still another aspect, provided herein is a method of treating obesity in a subject in need thereof, comprising administering to the subject an effective amount of a compound of the invention, e.g., compounds of Formulas I-XIV.

In still another aspect, provided herein is a method of treating atherosclerosis, comprising administering to a subject in need thereof an effective amount of a compound of the invention.

Atherosclerosis refers to the buildup of fats and cholesterol in and on artery walls (plaques), which can restrict blood flow. These plaques can also burst, triggering a blood clot. Although atherosclerosis is often considered a heart problem, it can affect arteries anywhere in the body. An animal model of atherosclerosis research is described in Laboratory Animals (2004) 38, 246-256.

The term “treat,” “treated,” “treating” or “treatment” includes the diminishment or alleviation of at least one symptom associated or caused by the state, disorder or disease being treated. In certain embodiments, the treatment comprises the induction of a disease, followed by the activation of the compound of the invention, which would in turn diminish or alleviate at least one symptom associated or caused by the protein kinase-associated disorder being treated. For example, treatment can be diminishment of one or several symptoms of a disorder or complete eradication of a disorder.

Atrial fibrillation is a type of cardiac arrhythmia where there is disorganized electrical conduction in the atria causing rapid uncoordinated contractions. These contractions can result in ineffective pumping of blood into the ventricle and a lack of synchrony. During atrial fibrillation, the atrioventricular node receives electrical impulses from numerous locations throughout the atria instead of only from the sinus node. This overwhelms the atrioventricular node into producing an irregular and rapid heartbeat. As a result, blood pools in the atria that increases a risk for blood clot formation. The major risk factors for atrial fibrillation include age, coronary artery disease, rheumatic heart disease, hypertension, diabetes, and thyrotoxicosis.

The term “subject” is intended to include organisms, e.g., prokaryotes and eukaryotes, which are capable of suffering from or afflicted with a disease, disorder or condition. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from obesity; cardiovascular diseases such as cardiac arrhythmia, cardiac ischemia, myocardial infarction, cardiomyopathy, and stroke; or hyperlipidemia, including hypertriglyceridemia,

The language “effective amount,” “pharmaceutically effective amount” or to “pharmaceutically acceptable amount” of the compound is that amount necessary or sufficient to treat or prevent a disorder, e.g., prevent the various morphological and somatic symptoms of a disease or condition described herein. In an example, an effective amount of a compound of the invention is the amount sufficient to treat obesity, cardiovascular disorders, or a related disorder, in a subject. The effective amount can vary depending on such factors as the size and weight of the subject, the type of illness, or the particular compound of the invention.

The triglyceride lowering efficacy of the compounds of the present invention can be determined in animal models according to the procedure described by Sidika et al in Journal of Lipid Research, 1992, 33, 1-7.

Efficacy of the compounds of the present invention in increasing adiponectin secretion in rodent models of obesity and human obese subjects can be determined according the procedures described by Itoh et al. in Arteroscler. Thromb. Vasc. Biol. 2007, 27, 1918-1925.

### Pharmaceutical Compositions

The compounds of the present invention are suitable as active agents in pharmaceutical compositions that are efficacious particularly for treating obesity, cardiovascular disorders, as well as related conditions. The pharmaceutical composition in various embodiments has a pharmaceutically effective amount of the present active agent along with other pharmaceutically acceptable excipients, carriers, fillers, diluents and the like. Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as is pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar, buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or is finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In one embodiment, the present invention relates to a pharmaceutical composition for the treatment of dyslipidemia, including hypertriglyceridemia, in mammals comprising an anti-dyslipidemia effective amount of a compound of the present invention and a pharmaceutically acceptable carrier. In one embodiment said mammals are human.

In one embodiment, the present invention relates to a pharmaceutical composition for the treatment of cardiovascular diseases, including cardiac arrhythmia, cardiac ischemia, myocardial infarction, cardiomyopathy, and stroke in mammals comprising an effective amount of a compound of the present invention to treat the said cardiovascular diseases and a pharmaceutically acceptable carrier. In an embodiment, the arrhythmia is an atrial fibrillation. In one embodiment said mammals are human

In one embodiment, the present invention relates to a pharmaceutical composition for the treatment of obesity, in mammals comprising an anti-obesity effective amount of a compound of the present invention and a pharmaceutically acceptable carrier. In one embodiment said mammals are human.

### Dosage

For administration to human patients, the total daily dose of the compounds of the invention is typically in the range 0.05 g to 12 g, e.g., 1 g to 12 g, depending, of course, on the mode of administration. In one embodiment the total daily dose is in the range 1 g to 10 g and in another embodiment the total daily dose is in the range 1 g to 6 g. In another embodiment, the daily dose is 0.05 g-6 g, 0.05 g-3 g, 0.05 g-1 g, or 0.05 g-0.2 g. The total daily dose may be administered in single or divided doses.

These dosages are based on an average human subject having a weight of about 65 kg to 70 kg. The physician will readily be able to determine doses for subjects whose weight falls outside this range, such as infants and the elderly.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the is active ingredient. The amount of the active ingredient is generally equal to the dosage of the active ingredient which would be administered to a subject or a convenient fraction of such a dosage such as, for example, one-half or one-third of such a dosage.

### Kits

Advantageously, the present invention also provides kits for use by a consumer for treating disease. The kits comprise a) a pharmaceutical composition comprising a compound of the invention and a pharmaceutically acceptable carrier, vehicle or diluent; and, optionally, b) instructions describing a method of using the pharmaceutical composition for treating the specific disease.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate to multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

One embodiment of the present invention relates to a kit comprising a unit dosage comprising a compound of the invention with instructions on how to use the kit and with provision for at least one container for holding the unit dosage form.

### Methods of Making

The salts of the invention can be prepared using any number of synthesis techniques known to the skilled artisan.

An example for the synthesis of the mono salt of piperazine with eicosapentaenoic acid (EPA) (Formula I) can be prepared as set forth below.

One equivalent of piperazine may be dissolved in an appropriate reaction inert solvent. The solvent may be polar such as water. As used herein, the expression “reaction inert solvent” refers to a solvent or a mixture of solvents that does not interact with starting materials, reagents, intermediates or products in a manner that adversely affects the yield of the desired product. Preferred solvents include methanol, ethanol, n-propanol, isopropanol, acetone, ethyl methyl ketone, diethyl ketone, methyl isobutylketone and acetonitrile. To this solution may be added a solution of one equivalent of EPA. Both piperazine and EPA are commercially available. The reaction mixture can be stirred at about ambient temperature to about the reflux temperature of the solvent being used for about two hours to about six hours. The mono salt of piperazine with EPA can be isolated from the mixture by methods well known to those skilled in the art, including according to the method of U.S. Pat. No. 7,973,073.

The di-salt of piperazine with EPA (Formula III) can be prepared according to the above procedure, but by using two equivalents instead of one equivalent of EPA. The mono salt of piperazine with docosahexaenoic acid (DHA) (Formula II) can be prepared as set forth below.

One equivalent of piperazine may be dissolved in an appropriate reaction inert solvent. The solvent may be polar such as water. Preferred reaction inert solvents include methanol, ethanol, n-propanol, isopropanol, acetone, ethyl methyl ketone, diethyl ketone, methyl isobutylketone and acetonitrile. To this solution may be added a solution of one equivalent of DHA. Both piperazine and DHA are commercially available. The reaction mixture can be stirred at about ambient temperature to about the reflux temperature of the solvent being used for about two hours to about six hours. The mono salt of piperazine with DHA can be isolated from the mixture by methods well known to those skilled in the art, including according to the method of U.S. Pat. No. 7,973,073.

The di-salt of piperazine with DHA (Formula IV) can be prepared according to the above procedure, but by using two equivalents instead of one equivalent of DHA.

The compound of Formula V can be prepared by adding a solution of one equivalent of compound XH to a solution of one equivalent of the compound of the Formula I. Suitable solvents include methanol, ethanol, n-propanol, isopropanol, acetone, ethyl methyl ketone, diethyl ketone, methyl isobutylketone and acetonitrile. The compound of Formula VI can be prepared by adding a solution of one equivalent of compound XH to a solution of one equivalent of the compound of the Formula II. Suitable solvents include methanol, ethanol, n-propanol, isopropanol, acetone, ethyl methyl ketone, diethyl ketone, methyl isobutylketone and acetonitrile.

The meglumine salt of EPA (Formula VII) can be prepared as set forth below.

One equivalent of meglumine may be dissolved in an appropriate reaction inert solvent. The solvent may be polar such as water. Preferred reaction inert solvents include methanol, ethanol, n-propanol, isopropanol, acetone, ethyl methyl ketone, diethyl ketone, methyl isobutylketone and acetonitrile. To this solution may be added a solution of one equivalent of EPA. Both meglumine and EPA are commercially available. The reaction mixture can be stirred at about ambient temperature to about the reflux temperature of the solvent being used for about two hours to about six hours. The meglumine salt of EPA can be isolated from the mixture by methods well known to those skilled in the art, including according to the method of U.S. Pat. No. 7,973,073.

The meglumine salt of DHA (Formula VIII) can be prepared as set forth below. One equivalent of meglumine may be dissolved in an appropriate reaction inert solvent. The solvent may be polar such as water. Preferred reaction inert solvents include methanol, ethanol, n-propanol, isopropanol, acetone, ethyl methyl ketone, diethyl ketone, methyl isobutylketone and acetonitrile. To this solution may be added a solution of one equivalent of DHA. Both meglumine and DHA are commercially available. The reaction mixture can be stirred at about ambient temperature to about the reflux temperature of the solvent being used for about two hours to about six hours. The meglumine salt of DHA can be isolated from the mixture by methods well known to those skilled in the art, including according to the method of U.S. Pat. No. 7,973,073.

Accordingly, in one aspect, provided herein is a method for the manufacture of a compound of Formula I or II, comprising: reacting one equivalent of piperazine, and one equivalent of eicosapentaenoic acid or docosahexaenoic acid, at a temperature between about 0° C. and about 60° C.

In another aspect, provided herein is a method for the manufacture of a compound of Formula III or IV, comprising: reacting one equivalent of piperazine, and two equivalents of eicosapentaenoic acid or docosahexaenoic acid, at a temperature between about 0° C. and about 60° C.

In still another aspect, provided herein is a method for the manufacture of a compound of Formula V or VI, comprising: reacting one equivalent of piperazine, one equivalent of eicosapentaenoic acid or docosahexaenoic acid, and one equivalent of a pharmaceutically acceptable acid, at a temperature between about 0° C. and about 60° C.

In another aspect, provided herein is a method for the manufacture of a compound of Formula IX or X, comprising: reacting one equivalent of ethylenediamine, and one equivalent of eicosapentaenoic acid or docosahexaenoic acid, at a temperature between about 0° C. and about 60° C.

In yet another aspect, provided herein is a method for the manufacture of a compound of Formula XI or XII, comprising: reacting one equivalent of ethylenediamine, and two equivalents of eicosapentaenoic acid or docosahexaenoic acid, at a temperature between about 0° C. and about 60° C.

In still another aspect, provided herein is a method for the manufacture of a compound of Formula XIII or XIV, comprising: reacting one equivalent of ethylenediamine, one equivalent of eicosapentaenoic acid or docosahexaenoic acid, and one equivalent of a pharmaceutically acceptable acid, at a temperature between about 0° C. and about 60° C.

In one embodiment of any of the above methods, the reaction is performed in the absence of a solvent.

### Example 1

### Preparation of Piperazine Eicopentaenoate R-(−)-Mandelate

A solution of piperazine (0.450 g, 5.22 mmol) in acetonitrile (30 mL, 600 mmol) was treated with a solution of (5Z,8Z,11Z,14Z,17Z)-icosa-5,8,11,14,17-pentaenoic acid (1.90 g, 6.27 mmol) in acetonitrile (30 mL, 600 mmol). The solution was stirred for 10 minutes, and then cooled at 0° C. Upon cooling a white precipitate formed. The suspension was treated drop wise over 30 minutes with a solution of R-(−)-mandelic acid (0.795 g, 5.22 mmol) in acetonitrile (24 mL, 460 mmol) and the mixture was stirred an additional 2.5 h at 0-5° C. The reaction mixture was filtered under nitrogen and the solid was washed with cold acetonitrile. The solid was quickly transferred to a round bottom flask and placed under high vacuum overnight. Yield was 2.40 g. The ¹H NMR spectrum, the ¹³C NMR spectrum and elemental analysis indicate the material is piperazine eicosapentaenoate R-(−)-mandelate.

Anal Calcd for C32HN2O5 plus 0.69% H20: C, 70.59; H, 8.96; N, 5.14. Found: C, 70.27; H, 8.84; N, 5.12.

¹H NMR (300 MHz, MeOD) d ppm 7.47 (m, 2H), 7.27 (m, 3H), 5.36 (m, 10H), 3.05 (s, 8H), 2.85 (m, 8H), 2.28 (t, J=7.45 Hz, 2H), 2.10 (m, 4H), 1.67 (m, 2H), 0.97 (t, J=7.54 Hz, 3H) ¹³C NMR (101 MHz, MeOD) d ppm 179.42, 178.49, 143.34, 132.93, 130.37, 129.88, 129.59, 129.42, 129.31, 129.27, 129.20, 129.08, 128.57, 128.33, 128.07, 76.10, 44.22, 35.18, 27.83, 26.70, 26.58, 26.42, 21.65, 14.81

### Example 2

### Bis[5Z,8Z,11Z,14Z,17Z)-icosa-5,8,11,14,17-pentaenoate]piperazine salt (EPA2-Pip)

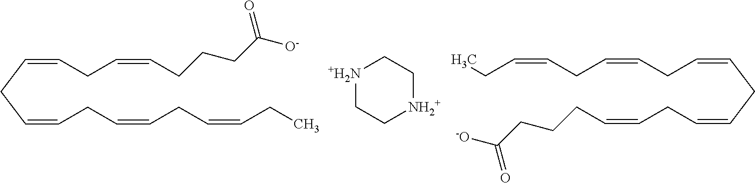

A stirred solution of piperazine (1.28 g, 14.9 mmol in acetonitrile (30 mL, 600 mmol) is treated dropwise with a solution of (5Z,8Z,11 Z,14Z,17Z)-icosa-5,8,11,14,17-pentaenoic acid (9.00 g, 29.8 mmol) in acetonitrile (73 mL, 1400 mmol) under N₂. After 30 minutes, the mixture is stored in the refrigerator overnight. The solid was collected by filtration and dried under hi-vac at RT overnight with P₂O₅. Yield=8.2 g of bis[(5Z,8Z,11Z,14Z,17Z)-icosa-5,8,11,14,17-pentaenoate]piperazine salt as a pink solid.

¹H NMR (300 MHz, MeOD) δ 0.99 (t, 6H) 1.68 (t, 4H) 2.04-2.18 (m, 3H) 2.11 (d, 5H) 2.25 (t, 4H) 2.85 (m, 17H) 3.07 (s, 9H) 5.27-5.45 (m, 20H); MS (ESI+) for C₂₀H₃₀O₂ m/z 303 (M+H)⁺; Anal Calcd for C₄₄H₇₀N₂O₄: C, 76.48; H, 10.21; N, 4.05. Found: C, 76.54; H, 10.09; N, 4.04.; MP=61-64° C.

### Example 3

### Ethane-1,2-diaminium di[5Z,8Z,11Z,14Z,17Z)-icosa-5,8,11,14,17-pentaenoate] (EPA2-EDA)

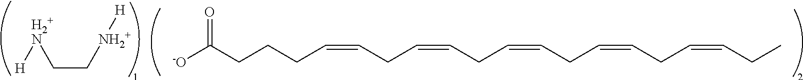

A mixture of (5Z,8Z,11 Z,14Z,17Z)-icosa-5,8,11,14,17-pentaenoic acid (10.50 g, 34.72 mmol) and ethylenediamine (1.10 mL, 16.5 mmol) in acetonitrile (215.8 mL, 4133 mmol) is stirred with ice bath cooling for 2 hrs and then stored in the refrigerator overnight. The solid is collected by filtration and dried under hi-vac at RT over P₂O₅ for 12 hrs. Yield=7.9 g of ethane-1,2-diaminium di[(5Z,8Z,11Z,14Z,17Z)-icosa-5,8,11,14,17-pentaenoate] as a brown solid. ¹H NMR (300 MHz, MeOD) δ 0.92 (t., 6H) 1.65 (m, 4H) 2.16 (m, 12H) 2.89 (m., 16H) 2.99 (s, 4H) 4.88 (s, 6H) 5.37 (br. s., 20H); MS (ESI+) for C₂₀H₃₀O₂ m/z 303 (M+H)₊; Anal Calcd for C₄₂H₆₈N₂O₄: C, 75.86; H, 10.31; N, 4.21. Found: C, 75.70; H, 10.25; N, 4.07. MP=30° C.

### Example 4

### Pharmacokinetics of Piperazine di-eicosapentaenoate

Oral pharmacokinetic parameters of piperazine di-eicosapentaenoate, prepared by the procedure described in Example 2, were determined in Sprague-Dawley rats. Piperazine di-eicosapentaenoate was administered by oral gavage as an aqueous solution in 0.5% carboxymethyl cellulose to 6 Sprague-Dawley rats, 3 males and 3 females. Rats were dosed at 40 mg/kg. Blood samples were obtained from each rat by jugular vein catheter. Samples were collected at 0.25, 0.5, 1, 2, 4, 8, 12, and 24 hours post dose. Blood samples were centrifuged to separate red blood cells and the resulting plasma samples were analyzed for eicosapentaenoic acid. Calculated pharmacokinetic parameters show below in Table 1 are mean values from 6 rats.

TABLE 1
Rat Oral Pharmacokinetic Parameters for
Piperazine di-eicosapentaenoate

| Analyte | EPA |
|---|---|
| Cₘₐₓ (μg/mL) | 28.22 |
| Tₘₐₓ (h) | 1.0 |
| AUC (0-24) | 134.85 |
| (μg * h/mL) | |

### Example 5

### Pharmacokinetics of Ethylene diamine di-eicosapentaenoate

Oral pharmacokinetic parameters of ethylene diamine di-eicosapentaenoate, prepared by the procedure described in Example 3, were determined in Sprague-Dawley rats. Ethylene diamine di-eicosapentaenoate was administered by oral gavage as an aqueous solution in 0.5% carboxymethyl cellulose to 6 rats, 3 males and 3 females. Rats were dosed at 40 mg/kg. Blood samples were obtained from each rat by jugular vein catheter. Samples were collected at 0.25, 0.5, 1, 2, 4, 8, 12, and 24 hours post dose. Blood samples were centrifuged to separate red blood cells and the resulting plasma samples were analyzed for eicosapentaenoic acid. Calculated pharmacokinetic parameters show below are mean values from 6 rats.

TABLE 2
Rat Oral Pharmacokinetic Parameters for
Ethylene diamine di-eicosapentaenoate

| Analyte | EPA |
|---|---|
| Cₘₐₓ (μg/mL) | 18.37 |
| Tₘₐₓ (h) | 2.0 |
| AUC (0-24) (μg * h/mL) | 275.29 |

1-26. (canceled)

27. A compound of the structural Formula III:

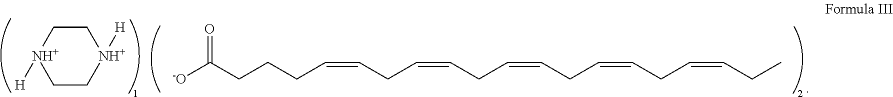

28. A compound of the structural Formula IV:

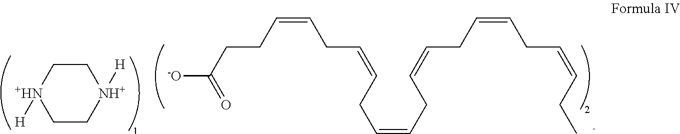

29. A pharmaceutical composition comprising a compound of any one of the above claims, and a pharmaceutically acceptable carrier.
